(19) United States
(12) Patent Application Publication
(10) Pub. No.: US 2019/0274517 A1
(43) Pub. Date: Sep. 12, 2019

(54) ELECTRIC ENDOSCOPE CONTROL DEVICE AND ELECTRIC ENDOSCOPE SYSTEM

(71) Applicant: OLYMPUS CORPORATION, Tokyo (JP)

(72) Inventor: Naoya HATAKEYAMA, Tokyo (JP)

(73) Assignee: OLYMPUS CORPORATION, Tokyo (JP)

(21) Appl. No.: 16/420,413

(22) Filed: May 23, 2019

Related U.S. Application Data
(63) Continuation of application No. PCT/JP2016/087928, filed on Dec. 20, 2016.

(51) Int. Cl. A61B 1/00; A61B 1/005

(52) U.S. Cl. CPC A61B 1/0002; A61B 1/0057

(57) ABSTRACT

An electric endoscope control device that changes a bending angle of a bending section of an endoscope by driving a motor according to an amount of manipulation, the electric endoscope control device includes: a storage that stores, for different types of operation state, a plurality of tables that each show the relationship between the amount of manipulation and the rotation angle of the motor, the electric endoscope control device configured to: perform an operation-state determining that determines the operation state of the bending section of the endoscope; and perform a drive-signal generating that generates a drive signal for the motor on a basis of one of the tables, which are stored in the storage, depending on the determined operation state.

### CROSS-REFERENCE TO RELATED APPLICATIONS

This is a continuation of International Application PCT/JP2016/087928, with an international filing date of Dec. 20, 2016, which is hereby incorporated by reference herein in its entirety.

### TECHNICAL FIELD

The present invention relates to an electric endoscope control device and an electric endoscope system.

### BACKGROUND ART

There is a known electric endoscope system that estimates an auxiliary torque required to match a bending angle with a target bending angle, on the basis of: bending characteristic information indicating the relationship between the amount of bending (hereinafter, simply referred to as bending angle) of a bending section and a manipulation torque for bending the bending section; and a constant parameter, and that corrects motor driving power for a drive motor on the basis of the estimated auxiliary torque, thus controlling the drive motor (for example, see PTL 1).

### CITATION LIST

### Patent Literature

{PTL 1} Publication of Japanese Patent No. 5396178

### SUMMARY OF INVENTION

One aspect of the present invention provides an electric endoscope control device that changes a bending angle of a bending section of an endoscope by driving a motor according to an amount of manipulation, the electric endoscope control device including: a storage that stores, for different types of operation state, a plurality of tables that each show the relationship between the amount of manipulation and the rotation angle of the motor, the electric endoscope control device configured to: perform an operation-state determining that determines the operation state of the bending section of the endoscope; and perform a drive-signal generating that generates a drive signal for the motor on a basis of one of the tables, which are stored in the storage, depending on the determined operation state.

Another aspect of the present invention provides an electric endoscope system including: an endoscope; and one of the above-described electric endoscope control devices.

According to this aspect, the endoscope may be provided with a pulley that is rotationally driven by the motor, a wire in which a pulling force for bending the bending section is produced by the pulley, and an angle sensor that detects a rotation angle of the pulley; and the operation-state determining unit may determine the operation state on the basis of the rotation angle detected by the angle sensor.

### BRIEF DESCRIPTION OF DRAWINGS

FIG. 1 is a view showing the overall configuration of an electric endoscope system according to a first embodiment of the present invention.

FIG. 2 is a view showing an example first table stored in a storage unit in an electric endoscope control device that is provided in the electric endoscope system shown in FIG. 1.

FIG. 3 is a view showing an example second table.

FIG. 4 is a view showing the overall configuration of an electric endoscope system according to a second embodiment of the present invention.

FIG. 5 is a view showing an example first table and an example second table that are stored in the storage unit in an electric endoscope control device of the electric endoscope system shown in FIG. 4.

FIG. 6 is a view showing a state in which a bending section is being operated in such a direction as to increase the curvature thereof, along the second table shown in FIG. 5.

FIG. 7 is a view for explaining switching from the second table shown in FIG. 5 to the first table, performed when the bending section is stopped.

FIG. 8 is a view for explaining switching from the first table shown in FIG. 5 to the second table, performed when the bending section is operated.

FIG. 9 is a view showing a state in which the bending section is being operated in such a direction as to decrease the curvature thereof, along the second table shown in FIG. 5.

### DESCRIPTION OF EMBODIMENTS

An electric endoscope control device 3 and an electric endoscope system 1 according to a first embodiment of the present invention will be described below with reference to the drawings.

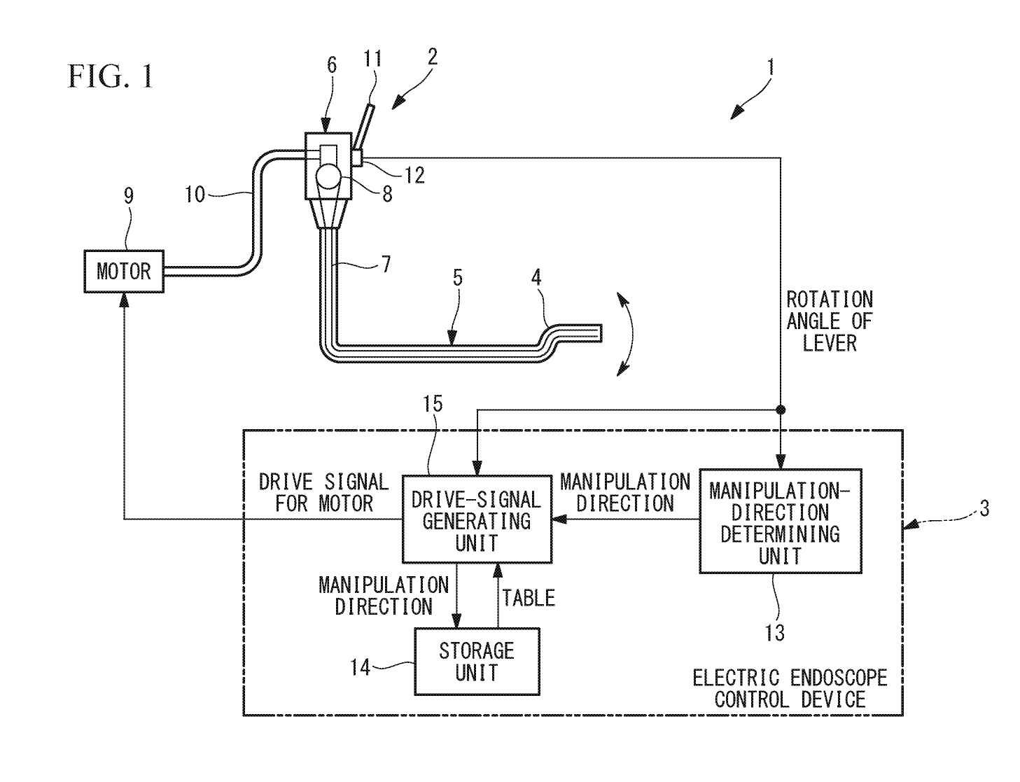

As shown in FIG. 1, the electric endoscope system 1 of this embodiment is provided with an electric endoscope (endoscope) 2 and an electric endoscope control device 3 that controls the electric endoscope 2.

The electric endoscope 2 is provided with: an elongated insertion portion 5 that has a bending section 4 at a distal end thereof; a manipulation unit 6 that is provided at a base end of the insertion portion 5; a wire 7 that is disposed along the longitudinal direction of the insertion portion 5 and that transmits a pulling force for bending the bending section 4; a pulley 8 that is disposed on the manipulation unit 6 and around which the wire 7 is wound; a motor 9; and a torque shaft 10 that transmits a torque of the motor 9 to the pulley 8.

The manipulation unit 6 is provided with: a lever 11 that is manipulated by an operator; and a potentiometer 12 that detects a rotation angle of the lever 11. The rotation angle of the lever 11 detected by the potentiometer 12 is sent to the electric endoscope control device 3 by a cable (not shown).

As shown in FIG. 1, the electric endoscope control device 3 of this embodiment is provided with: a manipulation-direction determining unit (operation-state determining unit) 13 that determines a manipulation direction (operation state) on the basis of the rotation angle (manipulation input signal) of the lever 11 detected by the potentiometer 12; a storage unit 14 that stores two kinds of tables corresponding to manipulation directions; and a drive-signal generating unit 15 that selects, from the storage unit 14, one of the tables depending on the manipulation direction determined by the manipulation-direction determining unit 13 and that generates a drive signal for the motor 9 corresponding to the rotation angle of the lever 11 on the basis of the selected table.

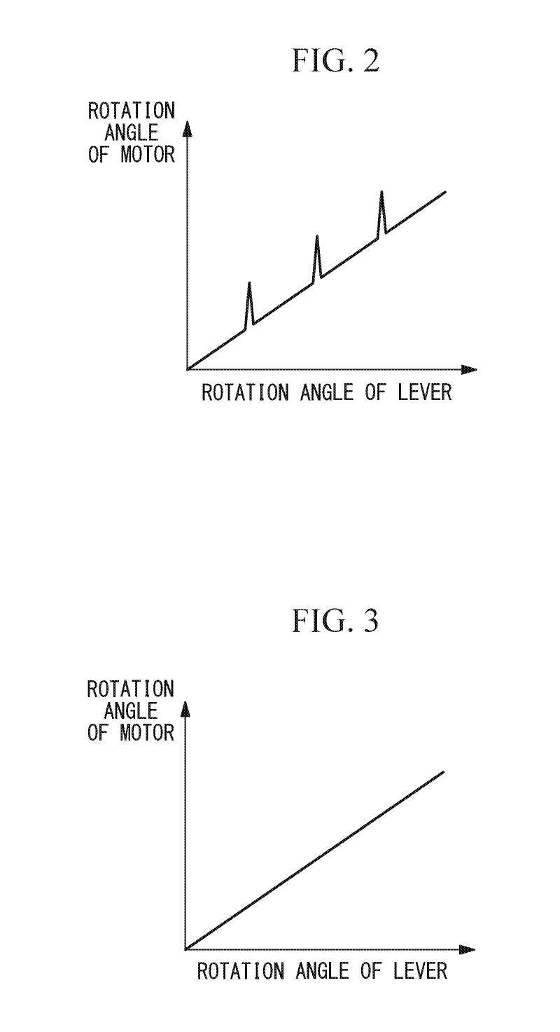

As shown in FIGS. 2 and 3, the two kinds of tables stored in the storage unit 14 can be two kinds of tables in each of which the rotation angle of the lever 11 is associated with a rotation-angle instruction for the motor 9. As shown in FIG. 2, the first table has a characteristic wherein, although the rotation angle of the lever 11 and the rotation-angle instruction for the motor 9 are linearly directly proportional to each other in most of the region, the instruction for the rotation angle of the motor 9 increases in a pulse-like manner at particular rotation angles of the lever 11 so as to rotate the motor 9 by a large amount momentarily.

The particular rotation angles of the lever 11 may be set by preliminarily detecting rotation angles at which the bending section 4 is stopped during the process in which the rotation angle of the lever 11 is increased.

As shown in FIG. 3, the second table has a simple linear-like characteristic obtained by removing the pulse-like parts from the first table.

The drive-signal generating unit 15 selects the first table when the manipulation direction of the lever 11, which is detected by the manipulation-direction determining unit 13, is a direction in which the curvature of the bending section 4 further increases, and selects the second table when the manipulation direction of the lever 11 is a direction in which the curvature of the bending section 4 decreases.

The operation of the thus-configured electric endoscope control device 3 and electric endoscope system 1 of this embodiment will be described below.

According to the electric endoscope system 1 of this embodiment, the insertion portion 5 of the electric endoscope 2 is inserted into a body, the distal end of the insertion portion 5 is disposed in the vicinity of an affected site, and the electric endoscope 2 is actuated, thereby making it possible to acquire an image of the affected site.

In this case, when the operator manipulates the lever 11 of the manipulation unit 6, the drive-signal generating unit 15 generates a drive signal for the motor 9 corresponding to the rotation angle of the lever 11 detected by the potentiometer 12, the motor 9 is driven according to the generated drive signal, a torque produced by the motor 9 is transmitted to the pulley 8 via the torque shaft 10, and the bending section 4, which is provided at the distal end of the insertion portion 5, is bent in a direction corresponding to the manipulation direction of the lever 11 by a pulling force produced in the wire 7 through rotation of the pulley 8, thus making it possible to change the field of view of the electric endoscope 2.

In this embodiment, when the bending section 4 is bent through manipulation of the lever 11, the manipulation-direction determining unit 13 determines the manipulation direction on the basis of the rotation angle of the lever 11 detected at time intervals by the potentiometer 12. In a case in which the bending section 4 continues to be bent in one direction, the manipulation-direction determining unit 13 determines that the manipulation direction is a direction in which the curvature of the bending section 4 increases.

Accordingly, the drive-signal generating unit 15 reads the first table from the storage unit on the basis of the determination result from the manipulation-direction determining unit 13.

Then, the drive-signal generating unit 15 reads, from the first table, a rotation-angle instruction for the motor 9 that corresponds to the rotation angle of the lever 11 of the manipulation unit 6, generates a drive signal, and outputs the drive signal to the motor 9. Accordingly, the motor 9 is rotationally driven.

In this case, according to this embodiment, because the first table has the characteristic in which the rotation-angle instruction for the motor 9 is increased in a pulse-like manner at the particular rotation angles of the lever 11, a rotation-angle instruction that is directly proportional to the rotation angle of the lever 11 is output to the motor 9 at most rotation angles of the lever 11, whereas a rotation-angle instruction that is momentarily increased in a pulse-like manner is output to the motor 9 when the rotation angle of the lever 11 reaches each of the particular rotation angles.

In this embodiment, because rotation angles at which the friction between the wire 7 and the insertion portion 5 increases, thereby stopping the operation of the bending section 4 even when the rotation angle of the lever 11 is changed, are preliminarily detected and are set as the particular rotation angles, when the rotation angle of the lever 11 reaches each of such particular rotation angles, the rotation-angle instruction for the motor 9 is temporarily increased in a pulse-like manner, thereby making it possible to temporarily increase the torque of the motor 9 and to produce, in the wire 7, a pulling force that exceeds the static friction force.

On the other hand, as a result of the determination of the manipulation-direction determining unit 13, if it is determined that the lever 11 is being manipulated in such a direction as to decrease the curvature of the bending section 4, the drive-signal generating unit 15 reads, from the second table read from the storage unit 14, a rotation-angle instruction for the motor 9 that corresponds to the rotation angle of the lever 11 of the manipulation unit 6, generates a drive signal, and outputs the drive signal to the motor 9.

Because the second table has the simple linear-like characteristic, and the friction between the wire 7 and the insertion portion 5 does not increase during the operation in this manipulation direction, a rotation-angle instruction for the motor 9 that is proportional to the rotation angle of the lever 11 is output to the motor 9, thereby making it possible to smoothly operate the motor 9 in a continuous manner.

Specifically, according to the electric endoscope control device 3 of this embodiment, when the bending section 4 is bent in such a direction as to increase the friction, the bending operation of the bending section 4 is prevented from being static due to the friction by the characteristic in the first table, which causes pulse-like changes at the particular angles, thus making it possible to easily prevent the bending operation of the bending section 4 from becoming jerky. On the other hand, regarding an operation of the bending section 4 in such a direction as not to increase the friction, there is an advantage in that, due to the characteristic in the second table, which causes no pulse-like changes, unnecessary variations in the bending operation are prevented, thus making it possible to smoothly perform the bending operation.

Next, an electric endoscope control device 21 and an electric endoscope system 20 according to a second embodiment of the present invention will be described below with reference to the drawings.

In the description of this embodiment, the same reference signs are assigned to portions whose configurations are common to those of the electric endoscope control device 3 and the electric endoscope system 1 according to the above-described first embodiment, and a description thereof will be omitted.

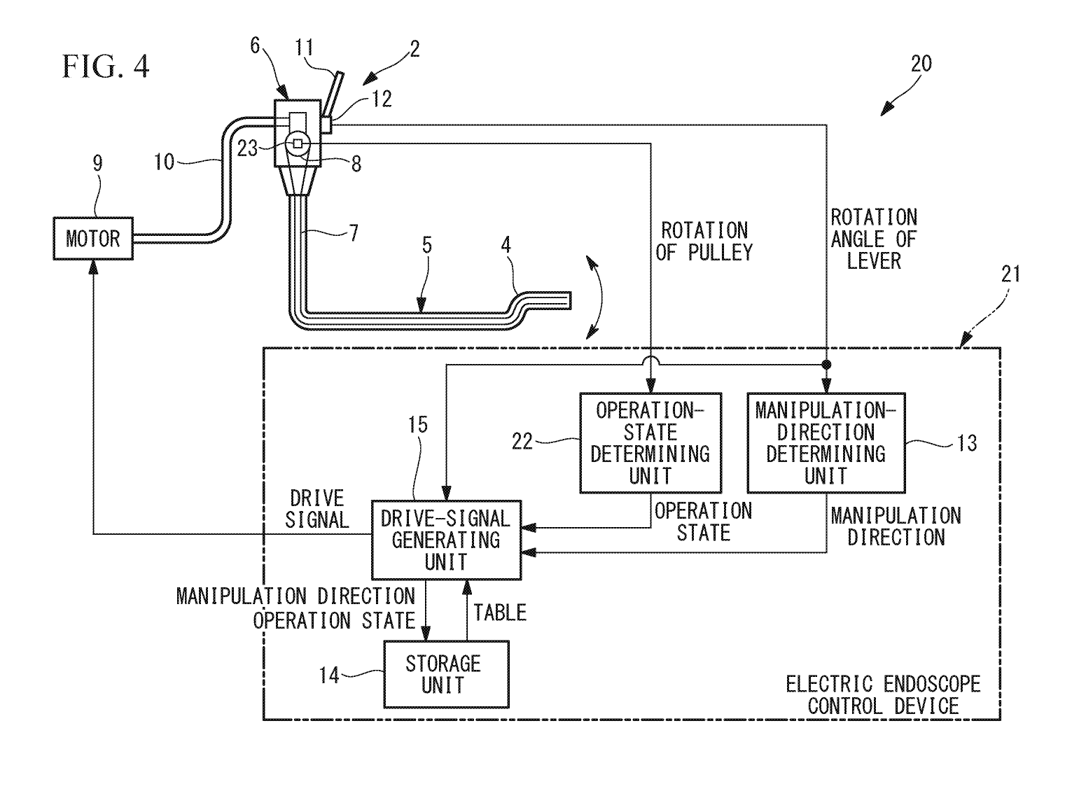

As shown in FIG. 4, the electric endoscope control device 21 of this embodiment differs from the electric endoscope control device 3 of the first embodiment in that an operation-state determining unit 22 is further provided.

Furthermore, the electric endoscope system 20 of this embodiment differs from the electric endoscope system 1 of the first embodiment in that the electric endoscope 2 that is provided in the electric endoscope system 20 is provided with a pulley-angle detecting unit (angle sensor) 23 that detects the rotation angle of the pulley 8, which is provided on the manipulation unit 6.

The operation-state determining unit 22 determines the operation state indicating whether the bending section 4 is static or not, on the basis of the rotation angle of the pulley 8 detected by the pulley-angle detecting unit 23.

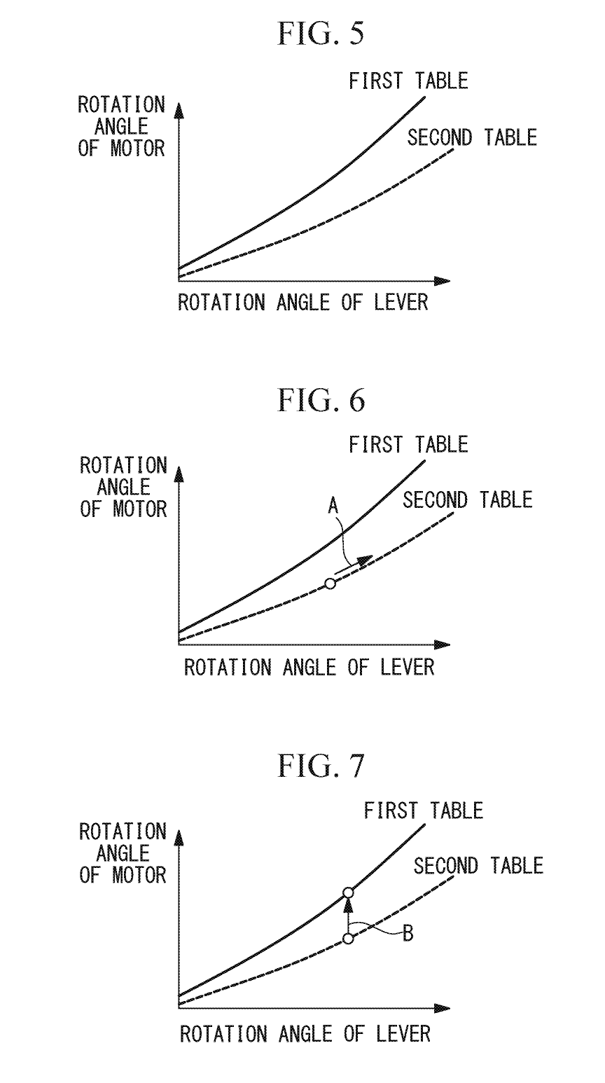

Furthermore, as shown in FIG. 5, the storage unit 14 stores a first table that is used when the bending section 4 is static and a second table that is used when the bending section 4 is being operated.

In this embodiment, the drive-signal generating unit 15 decides to use the first table or the second table on the basis of the determination result of the manipulation direction, which is output from the manipulation-direction determining unit 13, and the determination result of the operation state, which is output from the operation-state determining unit 22, and generates a rotation-angle instruction, i.e., a drive signal, for the motor 9 according to the characteristic prescribed in the table read from the storage unit 14.

More specifically, if the operation-state determining unit 22 determines that the bending section 4 is being operated, and the manipulation-direction determining unit 13 determines that the lever 11 is being manipulated in such a direction as to increase the curvature, the drive-signal generating unit 15 generates a drive signal so as to maintain the relationship between the rotation angle of the lever 11 and the rotation-angle instruction for the motor 9 that is based on the characteristic in the second table, as indicated by the arrow A in FIG. 6.

Then, if the operation-state determining unit 22 determines that the bending section 4 is static, the drive-signal generating unit 15 switches between the first table and the second table on the basis of the determination result of the manipulation direction from the manipulation-direction determining unit 13. Specifically, if the manipulation-direction determining unit 13 determines that the lever 11 is being manipulated in such a direction as to increase the curvature, the table to be used is switched to the first table, as indicated by the arrow B in FIG. 7.

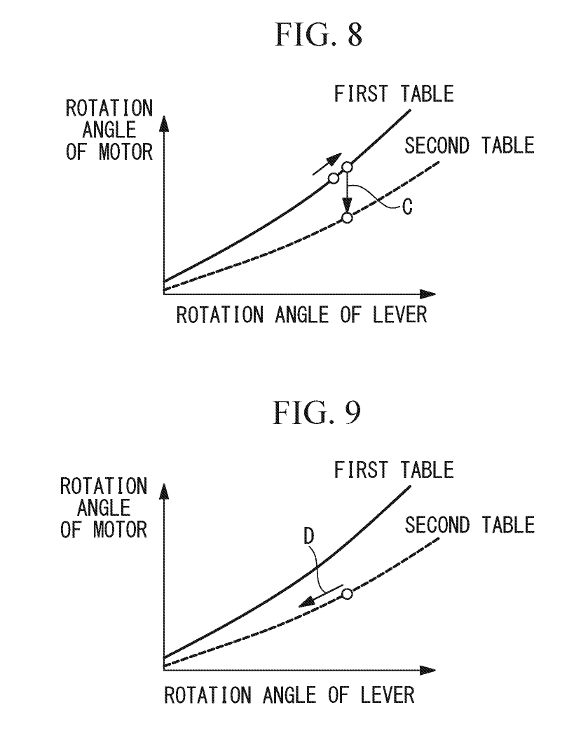

Then, through the switching from the second table to the first table, if the operation-state determining unit 22 determines that the bending section 4 is being operated, the table to be used is switched again to the second table, as indicated by the arrow C in FIG. 8.

On the other hand, if the manipulation-direction determining unit 13 determines that the lever 11 is being manipulated in such a direction as to decrease the curvature of the bending section 4, the drive-signal generating unit 15 selects the second table as the table to be used, as indicated by the arrow D in FIG. 9, and generates a drive signal according to the characteristic in the second table.

The operation of the electric endoscope control device 21 and the electric endoscope system 20 of this embodiment, which have been thus configured, will be described below.

According to the electric endoscope system 20 of this embodiment, the insertion portion 5 of the electric endoscope 2 is inserted into a body, the distal end of the insertion portion 5 is disposed in the vicinity of an affected site, and the electric endoscope 2 is actuated, thereby making it possible to acquire an image of the affected site.

In this case, the operator manipulates the lever 11 of the manipulation unit 6, thereby bending the bending section 4, which is provided at the distal end of the insertion portion 5, in a direction corresponding to the manipulation direction of the lever 11, and making it possible to change the field of view of the electric endoscope 2.

In this embodiment, when the bending section 4 is bent through manipulation of the lever 11, the manipulation-direction determining unit 13 determines the manipulation direction of the lever 11 on the basis of the rotation angle of the lever 11 detected at time intervals by the potentiometer 12. When the bending section 4 continues to be bent in one direction, the manipulation-direction determining unit 13 determines that the manipulation direction is a direction in which the curvature of the bending section 4 increases.

Furthermore, the motor 9 is driven through manipulation of the lever 11, the torque of the motor 9 is transmitted to the pulley 8 via the torque shaft 10, and the bending section 4, which is provided at the distal end of the insertion portion 5, is bent in a direction corresponding to the manipulation direction of the lever 11 by a pulling force produced in the wire 7 through rotation of the pulley 8, thereby making it possible to change the field of view of the electric endoscope 2. In this case, the operation state, which indicates whether the bending section 4 is being operated or is static depending on whether the pulley 8 is rotated, can be determined by a signal from the pulley-angle detecting unit 23, which is provided on the pulley 8.

As a result of determination of the operation-state determining unit 22, if it is determined that the bending section 4 is being operated, a rotation-angle instruction for the motor 9 is calculated according to the second table, which produces a lower torque, and, if it is determined that the bending section 4 is stopped, the table is switched depending on whether the manipulation direction determined by the manipulation-direction determining unit 13 is a direction in which the bending section 4 is bent by a larger amount. Specifically, if the bending section 4 is stopped, and the lever 11 is manipulated in such a direction as to bend the bending section 4 by a larger amount, the table is switched to the first table.

According to the first table, even at the same rotation angle of the lever 11, a rotation-angle instruction is output so as to rotate the motor 9 by a larger amount than in the second table; thus, the pulling force applied to the wire 7 can easily exceed the static friction between the wire 7 and the insertion portion 5. Specifically, because the stopped state of the bending section 4 is cancelled at the moment when the motor 9 is started to be operated according to the rotation-angle instruction based on the first table, the drive-signal generating unit 15 switches the table to the second table.

In this way, according to the electric endoscope control device 21 and the electric endoscope system 20 of this embodiment, as the tables, the first table, which is used when the bending section 4 is static, and the second table, which is used when the bending section 4 is being operated, are switched; effectively, when the bending section 4 is static, the torque is increased in a pulse-like manner, as in the table of FIG. 2 used in the first embodiment, thereby cancelling the state in which the bending section 4 is stopped due to the static friction, thus making it possible to prevent jerky operation of the bending section 4 and to smoothly operate the bending section 4.

Note that, in this embodiment, although the pulley-angle detecting unit 23 determines whether the bending section 4 is in a stopped state or in an operated state, instead of this, if a physical model indicating the friction state between the wire 7 and the insertion portion 5, which corresponds to the bending state of the bending section 4, can be built, it is also possible to determine the operation state of the bending section 4 by using the physical model. Accordingly, the pulley-angle detecting unit 23 becomes unnecessary, thus making it possible to simplify the structure of the electric endoscope 2.

Furthermore, instead of determining the operation state of the bending section 4 by means of the pulley-angle detecting unit 23, it is also possible to determine the operation state by using a shape sensor that detects the shape of the bending section 4. The operation state of the bending section 4 may also be detected by directly detecting the shape of the bending section 4 by using, as the shape sensor, an optical fiber sensor inserted into the electric endoscope 2, an endoscope position detecting unit (UPD) disposed outside the electric endoscope 2, or the like.

Furthermore, as described above, when the bending section 4 is static, the torque of the motor 9 is temporarily increased, thus producing a pulling force so as to overcome the static friction; however, if the bending section 4 is static with the distal end thereof being in contact with a tissue in the vicinity of an affected site, in some cases, it is better not to produce a large pulling force. In such cases, a pulling force to be produced may also be restricted to a predetermined value or less. For example, the electric endoscope system 20 is provided with: an angle-difference detecting unit that detects the difference between rotation angles at both ends of the torque shaft 10; and a restriction unit that restricts the operation of the motor 9 such that the difference between the rotation angles, which is detected by the angle-difference detecting unit, becomes less than a predetermined threshold. Then, the torque produced by the motor 9 may be restricted by the restriction unit such that the difference between the rotation angle of the motor 9 and the rotation angle of the pulley 8, which is detected by the angle-difference detecting unit, becomes less than the predetermined threshold.

Furthermore, in this embodiment, although the tables are switched depending on whether the manipulation direction is a direction in which the curvature of the bending section 4 increases, and the two tables are switched with each other irrespective of the bending direction of the bending section 4, instead of this, three or more tables may also be switched with each other.

For example, the rigidity of the torque shaft 10, which transmits the torque of the motor 9 to the pulley 8, in some cases, varies depending on the rotation direction thereof. In such cases, it is preferred to provide different tables depending on bending directions of the bending section 4, i.e., rotation directions of the torque shaft 10, and it is also possible to provide, separately for the rotation directions of the torque shaft 10, tables used when the manipulation direction is a direction in which the curvature of the bending section 4 increases.

As a result, the above-described embodiment leads to the following aspects.

One aspect of the present invention provides an electric endoscope control device that changes a bending angle of a bending section of an endoscope by driving a motor according to the amount of manipulation, the electric endoscope control device including: an operation-state determining unit that determines an operation state of the bending section of the endoscope; a storage unit that stores, for different types of the operation state, a plurality of tables that each show the relationship between the amount of manipulation and the rotation angle of the motor; and a drive-signal generating unit that generates a drive signal for the motor on the basis of one of the tables, which are stored in the storage unit, depending on the operation state determined by the operation-state determining unit.

According to this aspect, when an operator performs manipulation for bending the bending section of the endoscope, the motor is driven in accordance with the amount of manipulation. In this case, the operation state of the bending section is determined by the operation-state determining unit, a rotation angle of the motor in accordance with the amount of manipulation is obtained from one of the plurality of tables, which are stored in the storage unit, depending on the operation state, and the drive-signal generating unit generates such a drive signal for the motor as to achieve the rotation angle. Accordingly, even when the bending section is in a different operation state, it is possible to output a drive signal depending on the operation state and to smoothly operate the bending section.

In the above-described aspect, the operation-state determining unit may determine, as the operation state, whether or not the bending section of the endoscope is being operated in such a direction as to increase the curvature thereof.

In a case in which the bending section is being operated in such a direction as to increase the curvature, because the friction force produced on the endoscope tends to increase, it is necessary to increase the rotation angle of the motor in accordance with the amount of manipulation, compared with a case in which the bending section is being operated in such a direction as to decrease the curvature. By doing so, it is possible to switch the table, which shows the relationship between the amount of manipulation and the rotation angle of the motor, depending on whether the operation state is an operation state in which the friction force increases or an operation state in which the friction force does not increase, and to smoothly operate the bending section in any operation state.

Furthermore, in the above-described aspect, the operation-state determining unit may determine, as the operation state, whether or not the bending section of the endoscope is stopped.

In a case in which the bending section of the endoscope is stopped, it is necessary to apply such a force as to overcome a static friction force, and, in a case in which the bending section of the endoscope is being operated, it is sufficient to apply such a force as to overcome a dynamic friction force that is less than the static friction force. By doing so, with respect to the same amount of the manipulation, it is possible to actuate the motor on the basis of one of the different tables depending on whether the bending section is stopped or is being operated, and to smoothly operate the bending section in any operation state.

Furthermore, in the above-described aspect, one of the tables, which are stored in the storage unit, may show the relationship between the amount of manipulation and the rotation angle of the motor that is required for the bending section that is in a stopped state to be operated in such a direction as to increase the curvature thereof; and another one of the tables, which are stored in the storage unit, may show the relationship between the amount of manipulation and the rotation angle of the motor that is required for the bending section that is in an operated state to be continuously operated.

When the bending section of the endoscope is stopped, it is necessary to operate the motor with such a force as to overcome the static friction force, and, when the bending section of the endoscope is being operated, it is necessary to operate the motor with such a force as to overcome the dynamic friction force, which is less than the static friction force. By doing so, with respect to the same amount of the manipulation, the motor is rotated by a larger amount when the bending section is stopped than when the bending section is being operated, thus making it possible to smoothly operate the bending section in any operation state.

Another aspect of the present invention provides an electric endoscope system including: the endoscope; and one of the above-described electric endoscope control devices.

According to this aspect, the endoscope may be provided with a pulley that is rotationally driven by the motor, a wire in which a pulling force for bending the bending section is produced by the pulley, and an angle sensor that detects a rotation angle of the pulley; and the operation-state determining unit may determine the operation state on the basis of the rotation angle detected by the angle sensor.

By doing so, when the motor is rotationally driven, the pulley is rotated, and the bending section of the endoscope is bent by a pulling force produced in the wire wound around the pulley. The operation-state determining unit can easily determine the operation state of the bending section on the basis of the rotation angle of the pulley detected by the angle sensor, which is provided on the pulley.

Furthermore, the above-described aspect may further include a shape sensor that detects the shape of the bending section, wherein the operation-state determining unit may determine the operation state on the basis of the shape of the bending section detected by the shape sensor.

By doing so, the operation-state determining unit can directly determine the operation state of the bending section on the basis of the shape of the bending section detected by the shape sensor.

Furthermore, in the above-described aspect, the endoscope may be provided with a torque shaft that transmits power between the motor and the pulley; and one of the tables, which are stored in the storage unit, may be provided for each rotation direction of the torque shaft.

By doing so, even in a case in which the rigidity varies depending on the rotation direction of the torque shaft, it is possible to generate an appropriate drive signal by using a different table for each rotation direction of the torque shaft.

Furthermore, the above-described aspect may further include: an angle-difference detecting unit that detects the difference between rotation angles at both ends of the torque shaft; and a restriction unit that restricts an operation of the motor when the difference detected by the angle-difference detecting unit is a predetermined threshold or greater.

By doing so, it is possible to easily detect a case in which torques acting on both ends of the torque shaft become excessive and to restrict the motor from applying an excessive torque.

According to the present invention, an advantageous effect is afforded in that a bending section can be smoothly operated in response to manipulation performed by an operator.

### REFERENCE SIGNS LIST

1, 20 electric endoscope system
2 electric endoscope (endoscope)
3, 21 electric endoscope control device
4 bending section
7 wire
8 pulley
9 motor
10 torque shaft
13 manipulation-direction determining unit (operation-state determining unit)
14 storage unit
15 drive-signal generating unit
22 operation-state determining unit
23 pulley-angle detecting unit (angle sensor)

1. An electric endoscope control device that changes a bending angle of a bending section of an endoscope by driving a motor according to an amount of manipulation, the electric endoscope control device comprising:
a storage that stores, for different types of operation state, a plurality of tables that each show the relationship between the amount of manipulation and the rotation angle of the motor,
the electric endoscope control device configured to:
perform an operation-state determining that determines the operation state of the bending section of the endoscope; and
perform a drive-signal generating that generates a drive signal for the motor on a basis of one of the tables, which are stored in the storage, depending on the determined operation state.

2. The electric endoscope control device according to claim 1, wherein the operation-state determining determines, as the operation state, whether or not the bending section of the endoscope is being operated in such a direction as to increase a curvature thereof.

3. The electric endoscope control device according to claim 2, wherein the operation-state determining determines, as the operation state, whether or not the bending section of the endoscope is stopped.

4. The electric endoscope control device according to claim 2,
wherein one of the tables, which are stored in the storage, shows a relationship between the amount of manipulation and the rotation angle of the motor that is required for the bending section that is in a stopped state to be operated in such a direction as to increase the curvature thereof; and
another one of the tables, which are stored in the storage, shows the relationship between the amount of manipulation and the rotation angle of the motor that is required for the bending section that is in an operated state to be continuously operated.

5. An electric endoscope system comprising:
an endoscope; and
the electric endoscope control device according to claim 1.

6. The electric endoscope system according to claim 5,
wherein the endoscope is provided with a pulley that is rotationally driven by the motor, a wire in which a pulling force for bending the bending section is produced by the pulley, and an angle sensor that detects a rotation angle of the pulley; and
the operation-state determining determines the operation state on a basis of the rotation angle detected by the angle sensor.

7. The electric endoscope system according to claim 5, further comprising a shape sensor that detects the shape of the bending section,
wherein the operation-state determining determines the operation state on a basis of the shape of the bending section detected by the shape sensor.

8. The electric endoscope system according to claim 6,
wherein the endoscope is provided with a torque shaft that transmits power between the motor and the pulley; and
one of the tables, which are stored in the storage, is provided for each rotation direction of the torque shaft.

9. The electric endoscope system according to claim 8, configured to:
perform an angle-difference detecting that detects a difference between rotation angles at both ends of the torque shaft; and
restrict an operation of the motor when the detected difference is a predetermined threshold or greater.
